(19) United States
(12) Patent Application Publication
(10) Pub. No.: US 2025/0114342 A1
(43) Pub. Date: Apr. 10, 2025

(54) APIXABAN ORAL LIQUID DOSAGE FORMS

(71) Applicant: Shilpa Medicare Limited, RAICHUR (IN)

(72) Inventors: GANESHCHANDRA SONAVANE, Ahmedabad (IN); SHANTARAM PAWAR, Ahmedabad (IN); MALAY PATEL, Ahmedabad (IN)

(73) Assignee: Shilpa Medicare Limited, RAICHUR (IN)

(21) Appl. No.: 18/905,163

(22) Filed: Oct. 3, 2024

(30) Foreign Application Priority Data
Oct. 4, 2023 (IN) 202341066554
Feb. 28, 2024 (IN) 202441014414

(51) Int. Cl. A61K 31/4545; A61K 9/00; A61K 47/02; A61K 47/10; A61K 47/14; A61K 47/24

(52) U.S. Cl. CPC A61K 31/4545; A61K 9/0053; A61K 47/02; A61K 47/10; A61K 47/14; A61K 47/24

(57) ABSTRACT

The present invention discloses liquid dosage forms of apixaban in the form of aqueous & non-aqueous solutions for oral administration. The formulations of the present invention can include higher amount of apixaban with lower amount of solvent and/or vehicles.

### FIELD OF THE INVENTION

The present invention relates to the oral liquid dosage forms of a factor Xa (FXa) inhibitor drug, Apixaban. The oral liquid dosage forms of the present invention are solution dosage forms, which includes aqueous & non-aqueous solutions.

### BACKGROUND OF THE INVENTION

Apixaban, a factor Xa (FXa) inhibitor, is chemically described as 1-(4-methoxyphenyl)-7-oxo-6-[4-(2-oxopiperidin-1-yl)phenyl]-4, 5, 6, 7-tetrahydro-1H-pyrazolo[3, 4-c]pyridine-3-carboxamide. Its molecular formula is C₂₅H₂₅N₅O₄, which corresponds to a molecular weight of 459.5. Apixaban has the following structural formula:

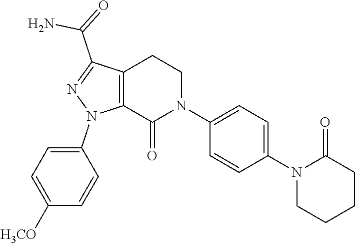 [SMILES: COc1ccc(-n2nc(C(N)=O)c3c2C(=O)N(c2ccc(N4CCCCC4=O)cc2)CC3)cc1]

Apixaban has been approved by the Unites States Food and Drug Administration (“USFDA”) as 2.5 mg & 5 mg oral tablets (ELIQUIS) to reduce the risk of stroke and systemic embolism in patients with nonvalvular atrial fibrillation. ELIQUIS is also indicated for the prophylaxis of deep vein thrombosis (DVT), which may lead to pulmonary embolism (PE), in patients who have undergone hip or knee replacement surgery. ELIQUIS is also indicated for the treatment of DVT and PE, and for the reduction in the risk of recurrent DVT and PE following initial therapy. The recommended daily dose of ELIQUIS for different indications varies from 5 mg to 20 mg.

The ELIQUIS tablets are administered orally as whole tablets by swallowing. However, for patients who are unable to swallow the whole tablets 5 mg and 2.5 mg ELIQUIS tablets may be crushed and suspended in water, 5% dextrose in water (D5W), or apple juice, or mixed with applesauce and promptly administered orally. Alternatively, ELIQUIS tablets may be crushed and suspended in 60 mL of water or D5W and promptly delivered through a nasogastric tube. Crushed ELIQUIS tablets are stable in water, D5W, apple juice, and applesauce for up to 4 hours. Thus, a liquid formulation is important for administration of apixaban in the pediatric population and adults who are unable to swallow a solid dosage form.

Low aqueous solubility of apixaban (0.04 mg/mL) is a major hurdle for the development of a liquid formulation of apixaban. The inventors of the International Application filed by Bristol Myers and Squibb Company & Pfizer Inc. having Publication No. WO 2014/052678 (hereinafter referred as “the WO'678 publication”) conducted extensive solubility studies to identify a vehicle that supports a low dose concentration.

Liquid formulations disclosed and taught by the WO'678 publication comprises apixaban and a vehicle that comprises non-ionic surfactant, ionic surfactant, hydrophilic polymer, polyhydric alcohol, polyethylene glycol, and carbohydrate. These excipients are identified as solubilizers by the inventors of the WO'678 publication and they have also identified that at least two of these solubilizers are required to achieve the desired solubility of apixaban. More particularly, the WO'678 publication teaches a liquid formulation comprising apixaban and a vehicle, wherein the vehicle comprises water, and preferred solubilizers viz. glycerin, propylene glycol, polyethylene glycol, polysorbate, povidone, sorbitol, sodium lauryl sulfate, copovidone, poloxamer, fructose and sucrose. Thus, the WO'678 publication essentially teaches aqueous apixaban solutions wherein the water content of the vehicle is about 20% w/w to about 30% w/w. Thus, the WO'678 publication does not teach non-aqueous apixaban liquid formulations. Further, too many types of solubilizers are used and the dosage form comprises many excipients, which may not be advisable for pediatric patients.

It was determined that a concentration of 0.4 mg/mL of apixaban in an oral liquid formulation adequately supports a desired dosage range of 0.04 mg to 5.0 mg with acceptable volumes ranging, for example, between 0.10 mL and 12.5 mL, which can be accurately measured and conveniently administered in the target patient population. However, an oral liquid formulation of the WO'678 publication results into relatively high dose volumes (˜25 mL).

Patients undergoing hip surgery and geriatric patients who are usually unwilling and/or unable to swallow high dose volumes, high dose volume cause discomfort. Discomfort while swallowing, especially for bedridden patients are the most important parameters governing patient compliance. Liquid medicaments intended for the oral administration are advantageous to be administrated in small dose volume of less than 10 mL, especially for geriatric, pediatric patients and patients undergoing hip surgery who are bedridden. Small dose volume of liquid medicaments are also advantageous for patients who usually receive their medication through feeding tubes. The inventors of the International Application filed by Dafechem Limited having Publication No. WO 2022/123074 (hereinafter referred as “the WO'074 publication”) attempted a liquid formulation for oral delivery of apixaban comprising i) apixaban in high concentrations, ii) more than 35% (w/v) propylene glycol based on the total formulation and iii) optionally other pharmaceutical acceptable excipients, wherein the formulation is suitable for small dose volume administration.

The small dose volume described by the WO'074 publication is less than 10 mL, preferably equal to or less than 5 mL, more preferably equal to or less than 4 mL, more preferably equal to or less than 3 mL, even more preferably equal to or less than 2 mL, preferably equal to or less than 1 mL. The amount of propylene glycol taught in the WO'074 publication is more than 35% (w/v) propylene glycol based on the total formulation. Preferably, the amount of propylene glycol taught by the WO'074 publication is more than 40% (w/v) based on the total formulation, more preferably more than 45% (w/v) propylene glycol based on the total formulation, even more preferably more than 50% (w/v) propylene glycol based on the total formulation.

The inventors of the WO'074 publication were concerned about the low-dose volumes of the apixaban formulation and therefore they prepared concentrated solutions of apixaban. To achieve this, they used higher amount of propylene glycol, i.e. at least 35% (w/v) and higher temperature (90° C.) to dissolve apixaban. Following this, most concentrated solution of up to 10 mg/mL apixaban in at least 35% (w/v) propylene glycol was prepared. Table-1 of the WO'074 publication summarizes results of the solubility testing studies conducted with various amounts of apixaban against various amounts of solvents and temperature conditions used. Table-1 teaches that when higher amount of apixaban is used, apixaban remains insoluble at 20° C. but gets dissolved at 40° C. in the same amount of solvents (i.e. 40% propylene glycol+60% glycerol). Further, Table-1 shows that even with the use of 100% propylene glycol, 2 mg of apixaban remains partially soluble at 20° C.

Thus, the WO'074 publication does not teach apixaban liquid formulations comprising less than 35% (w/v) propylene glycol. The apixaban liquid formulation of the WO'074 publication is essentially a non-aqueous formulation.

A yet another prior art document, which is a Chinese Patent Application No. CN 113384526 (hereinafter referred as “the CN'526 application”) teaches apixaban oral solution preparation. The apixaban oral solution taught by the CN'526 application is again an aqueous formulation and contains solubilizers selected from one or more of diethylene glycol monoethylether, caprylic acid capric acid polyethylene glycol glyceride, and 15-hydroxystearic acid polyethylene glycol ester.

A yet another prior art document, which is a United States Application Publication No. US 2023/0285368 assigned to Slayback Pharma LLC (hereinafter referred as “the US'368 publication”) teaches apixaban aqueous oral formulations that comprises 35%-40% w/w water and 0.1% w/w to 7% w/w polysorbate. The apixaban oral solution taught in the US'368 publication is free of polyethylene glycol. However, the said formulation comprises 0.4 mg/mL apixaban.

A yet another prior art document, which is a U.S. Pat. No. 11,833,138 assigned to Tap Pharmaceuticals teaches a liquid pharmaceutical formulation, suitable for oral administration, that comprises a therapeutically effective amount of apixaban; and an amount of glycerol sufficient to result in from 0.75 mg/ml to 1.75 mg/ml of the apixaban being in the solution phase of the formulation, at 20 hours after the formulation's manufacture when the formulation is kept at 25° C.±2° C., wherein the formulation is free of added: polysorbate, poloxamer, polyoxyethylene castor oil, a polyoxyglyceride, vitamin E polyethylene glycol succinate, macrogol 15 hydroxy stearate, sodium lauryl sulfate, and docusate sodium; povidone, hydroxypropyl cellulose, and hydroxypropyl methylcellulose; propylene glycol, sorbitol, and mannitol; and fructose, sucrose, and lactose.

A yet another prior art document, which is an international publication having no. WO 2023/218482 filed by Syri Research Private Limited teaches apixaban oral suspensions comprising 1 mg/mL apixaban and a solubilizer wherein solubilizer comprises a polymer and a surfactant. In the said suspension, apixaban has a particle size distribution D (90) less than about 20 microns.

A yet another International Application filed by Pharma-Data S.A. having Publication No. WO 2023/072967 (hereinafter referred as “the WO'967 publication”) discloses buffered oral aqueous apixaban suspension comprising micronized apixaban and having a pH of 1.5-6.5. The aqueous suspension of the WO'967 publication is devoid of emulsifier, dispersant, surfactant and solubilizer.

A yet another Chinese Patent Application No. CN 109010273 (hereinafter referred as “the CN'273 application”) describes a nano-suspension of apixaban with improved solubility and dissolution rate, with a particle size of less than 500 nm, further comprising stabilizer. The prepared apixaban nano-suspension uses glycocholate sodium and polyvinyl lactam-polyvinyl acetate-polyethylene glycol graft copolymerization (Soluplus) as a combined stabilizing agent.

In view of the foregoing paragraphs, it can be seen that the scientists have encountered difficulties while preparing apixaban oral liquid dosage forms (solution & suspension dosage forms) and have attempted below mentioned solutions:
1. Preparation of oral solution with lower amount of apixaban due to less solubility;
2. Use of large numbers of solubilizers including surfactants and organic solvents to solubilize apixaban;
3. High dose volume due to less solubility of apixaban in the vehicle;
4. Use of propylene glycol in higher amount to dissolve apixaban;
5. Use of glycerin in larger amount to dissolve apixaban;
6. Preparation of nano-suspension to achieve dissolution and bio-availability; and
7. Use of different stabilizing agents.

In view of the difficulties documented in the literature and its solution provided, it can be summarized that there is still an existing need for the development of an oral liquid dosage form of apixaban either in the form of a solution or in the form of a suspension. Such a liquid dosage form should address the issues of low solubility of apixaban, use of less numbers of excipients, use of excipients, which are safe at least for pediatric patients, use of excipients in acceptable amount for administration to all types of patient population, e.g. pediatric and geriatric.

The present inventors have therefore, through their extensive experimental studies designed a non-aqueous and aqueous oral solution of apixaban, which is safe and patient compliant. Said non-aqueous and aqueous solutions contain higher amount of apixaban dissolved in an acceptable and minimum required amount of solvents and/or vehicles. Further, as opposed to the prior art documents, said non-aqueous and aqueous solutions do not contain higher amount of propylene glycol (i.e. at least 35% w/v) and glycerin (i.e. more than 400 mg/mL and up to 1260 mg/mL). The present inventors have also attempted to prepare aqueous solutions of apixaban in the absence of surfactants (ionic and/or non-ionic), and solubilizers such as diethylene glycol monoethylether, caprylic/capric acid polyethylene glycol glyceride and 15-hydroxystearic acid polyethylene glycol ester. Further, with the help of the formulations of the present invention, aqueous solutions can be prepared which contains 0.4 mg/mL to 1 mg/mL apixaban.

### OBJECTS OF THE INVENTION

It is therefore an object of the present invention to provide a non-aqueous and aqueous oral solution of apixaban, which overcomes the problems of less aqueous solubility of apixaban.

A yet another object of the present invention is to provide a non-aqueous and aqueous oral solution of apixaban that comprises higher amount of apixaban dissolved in the solution, e.g. 1 mg/mL. Such an apixaban oral solution can be used in lower dose amounts and patient discomforts can be minimized.

A yet another object of the present invention is to provide a non-aqueous and aqueous oral solution of Apixaban, which comprises less than 35% w/v propylene glycol based on the total weight/volume of the formulation. A yet another object of the present invention is to provide an aqueous oral solution of Apixaban, which comprises less than 1260 mg/mL glycerin based on the total weight/volume of the formulation.

A yet another object of the present invention is to provide an aqueous oral solution of apixaban, which is devoid of one or more excipients selected from surfactants, and solubilizers selected from diethylene glycol monoethylether, caprylic/capric acid polyethylene glycol glyceride and 15-hydroxystearic acid polyethylene glycol ester.

A yet another object of the present invention is to provide an aqueous oral solution of apixaban, which contains added glycerin, propylene glycol, polyethylene glycol and optionally ethanol.

A yet another object of the present invention is to provide an aqueous oral solution of apixaban, which comprises a cyclodextrin, or a derivative thereof and/or a polymer.

The cyclodextrin, or a derivative thereof comprises at least one of α (alpha)-cyclodextrin, β (beta)-cyclodextrin, γ (gamma)-cyclodextrin, and a combination thereof.

A yet another object of the present invention is to provide a process for preparing apixaban formulation of the present invention.

A yet further object of the present invention is to provide a non-aqueous and aqueous oral solution having similar bioavailability and pharmacokinetic properties to ELIQUIS® (apixaban) tablets.

In a yet further object, the present invention provides a method for treating a thromboembolic disorder comprising administering to a patient in need thereof a therapeutically effective amount of an apixaban formulation of the present invention.

In a yet further object, the present invention provides an apixaban formulation of the present invention for use in treating a thromboembolic disorder.

In a yet another object, the present invention provides use of an apixaban formulation of the present invention in the treatment of a thromboembolic disorder.

In a yet another object, the present invention provides use of an apixaban formulation of the present invention in the preparation of a medicament for use in treating a thromboembolic disorder.

A yet another object of the present invention is to provide a method of use of an apixaban formulation of the present invention for oral administration which can be adjustable to the prescribed dosing scheme and can be administered in multiple dosing times.

### DETAILED DESCRIPTION OF THE INVENTION

Disclosed herein are oral liquid formulations of apixaban. The oral liquid formulations, preferably are essentially in the form of non-aqueous and aqueous solutions. The non-aqueous and aqueous solution of apixaban contains higher amount of apixaban per mL and overcomes the solubility problems thereof.

### Non-Aqueous Oral Solutions of Apixaban

In some embodiments, the non-aqueous solution of apixaban comprises one or more excipients selected from the group of solvents, and vehicles. In some embodiments, the non-aqueous solution of apixaban optionally comprises a viscosity modifying agent, a sweetening agent, and/or a favoring agent.

In some embodiments, the non-aqueous solution of apixaban comprises a solvent comprising about 1% w/v to about 35% w/v Labrasol® or Labrasol® ALF (USP NF name: Caprylocaproyl Polyoxyl-8 glycerides; EP name: Caprylocaproyl macrogol-8 glycerides; preferred substance name (FDA): PEG-8 CAPRYLIC/CAPRIC GLYCERIDES) based on the total weight/volume of the formulation; a vehicle comprising one or more selected from about 10% w/v to about 50% w/v phosphatidylcholine based on the total weight/volume of the formulation, about 10% w/v to less than about 35% w/v propylene glycol based on the total weight/volume of the formulation, about 0.1% w/v to about 5% w/v ethanol based on the total weight/volume of the formulation, and medium chain triglycerides in a quantity sufficient to make the % weight of the formulation 100% or volume of the formulation to 1 mL. In some embodiments, the non-aqueous solution of apixaban comprises mixture of phosphatidylcholine (a phospholipid), propylene glycol and ethanol (e.g. Phosal® 50 PG) as vehicle. In some embodiments, the non-aqueous solution of apixaban comprises mixture of Phosal® 50 PG and medium chain triglycerides as vehicle. In some embodiments, the non-aqueous solution of apixaban optionally comprises a sweetening agent and/or a flavoring agent.

The non-aqueous oral solution of the present invention may include an appropriate amount of a viscosity modifying agent, which is effective to stabilize the pharmaceutically active agent within the non-aqueous composition or used to increase the viscosity of the solution. A viscosity modifying agent of the present invention may include but not restricted to one or more of silicone dioxide and polyacrylate and polyacrylate copolymers such as methacrylic acid-methyl methacrylate copolymer (1:1. 1:2 etc.; e.g. Eudragit® L-100, Eudragit® S-100 etc.).

Optionally other viscosity modifying agents include celluloses and cellulose derivatives for example methyl-, ethyl- and propyl celluloses; hydroxyalkyl-celluloses, hydroxyl propyl celluloses, hydroxylpropylalkyl celluloses, carboxymethyl cellulose or its salt and the like including xanthan gum, polyvinyl resins, polyethylene glycol, polyethylene oxide, sorbitol, sucrose, xylitol, dextrose, fructose, maltitol, sugar, sodium alginate and the like.

The viscosity modifying agents used either single or in combinations in the range of about 0.001% to about 20% by weight relative to the total weight/volume of the composition.

Thus, in a preferred embodiment, the present invention provides a non-aqueous oral solution of apixaban comprising Labrasol® or Labrasol® ALF (USP NF name: Caprylocaproyl Polyoxyl-8 glycerides; EP name: Caprylocaproyl macrogol-8 glycerides; preferred substance name (FDA): PEG-8 CAPRYLIC/CAPRIC GLYCERIDES); a mixture of phosphatidylcholine, propylene glycol and ethanol (Phosal® 50 PG); and a viscosity modifying agent selected from silicon dioxide and methacrylic acid-methyl methacrylate copolymer (1:1. 1:2 etc.; e.g. Eudragit® L-100, Eudragit® S-100 etc.). The non-aqueous solution of apixaban further comprises a sweetening agent and/or a flavoring agent.

In a more preferred embodiment, the present invention provides a non-aqueous oral solution of apixaban consisting essentially of about 0.5 mg/mL to about 10 mg/mL apixaban, less than about 35% w/v propylene glycol, about 15% w/v to about 20% w/v Caprylocaproyl Polyoxyl-8 glycerides (Labrasol®, Labrasol® ALF), about 35% w/v phosphatidylcholine, about 1% w/v ethanol, medium chain triglycerides in a quantity sufficient to make up the volume of the formulation 1 mL, and about 0.2% w/v methacrylic acid-methyl methacrylate copolymer.

In a most preferred embodiment, the present invention provides a non-aqueous oral solution of apixaban consisting of about 0.5 mg/mL to about 10 mg/mL apixaban, less than about 35% w/v propylene glycol, about 15% w/v to about 20% w/v Caprylocaproyl Polyoxyl-8 glycerides (Labrasol®, Labrasol® ALF), about 35% w/v phosphatidylcholine, about 1% w/v ethanol, medium chain triglycerides in a quantity sufficient to make up the volume of the formulation 1 mL, about 0.2% w/v methacrylic acid-methyl methacrylate copolymer (1:2), a sweetener and a flavor.

Without intending of being bound by any theory, it is believed that the viscosity modifying agent increases the viscosity of the apixaban solution and thus helps retard the dissolution of apixaban in different dissolution media. Thus, in the present invention, the viscosity modifying agent may also alternatively be known as a dissolution retarding agent.

In some embodiments, the non-aqueous solution of apixaban comprises less than 35% w/v propylene glycol based on total weight/volume of the formulation. In some embodiments, the non-aqueous solution of apixaban comprises less than 30% w/v, less than 25% w/v, less than 20% w/v or less than 15% w/v propylene glycol based on the total weight/volume of the formulation. In some of the preferred embodiments, the non-aqueous solution of apixaban comprises about 15% w/v to about 30% w/v propylene glycol based on total weight/volume of the formulation. In some of the more preferred embodiments, the non-aqueous solution of apixaban comprises about 20% w/v to about 25% w/v propylene glycol based on the total weight/volume of the formulation.

In some embodiments, level of the 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxylic acid impurity in the non-aqueous oral solutions of apixaban of the present invention is less than about 0.5% w/w, preferably less than about 0.3% w/w, more preferably less than about 0.2% w/w, and most preferably less than about 0.1% w/w. In some of the preferred embodiments of the present invention, level of the 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxylic acid impurity in the non-aqueous oral solutions of apixaban of the present invention is equal to or less than about 0.05% w/w. In some embodiments, the total impurities present in the apixaban non-aqueous oral solution of the present invention are less than about 5% w/w or less than about 3% w/w or less than about 1% w/w or less than about 0.5% w/w.

### Aqueous Solutions of Apixaban

In some embodiments, the aqueous solution of apixaban comprises one or more solubility enhancers, e.g. a cyclodextrin, or a derivative thereof such as β (beta)-cyclodextrin, which without limitation includes hydroxypropyl betacyclodextrin (HPBCD), sulfobutylether-beta-cyclodextrin (SBECD) and/or a polymer such as povidone/crospovidone. Without intending of being bound by any theory, it is believed that the cyclodextrin, or a derivative thereof forms complex and helps dissolve the active ingredient in the liquid media. In a preferred embodiment, the concentration of the solubility enhancing agent ranges from about 0.1% to 80%, based on total weight/volume of the composition.

A polymer as referred herein is a compound of high molecular weight derived by the addition of many smaller units and which has a strong affinity for water. Suitable polymers can be selected from the group consisting of povidone, hydroxypropyl cellulose, or hydroxypropyl methylcellulose. In a preferred embodiment, the concentration of polymer ranges from about 0.5% to 20%, based on total weight/volume of the composition.

In addition to the solubility enhancing agents, in some embodiments, the aqueous solution of apixaban further comprises one or more excipients selected from the group of preservatives, solvents, pH adjusting agents/buffering agents, and vehicles. In some embodiments, the aqueous solution of apixaban optionally comprises a sweetening agent, and/or a favoring agent.

In some embodiments, the aqueous solution of apixaban comprises a solvent comprising glycerin, propylene glycol, polyethylene glycol, and optionally an ethanol or any combination thereof; a vehicle comprising water; a preservative comprising methyl paraben or its salt, ethyl paraben or its salt, propyl paraben or its salt, benzoic acid or its salt and any combination thereof; a pH adjusting agent and/or a buffering agent which helps adjust pH of the solution to about 4.0. In some embodiments, the aqueous solution of apixatan comprises mixture of glycerin, propylene glycol and polyethylene glycol as solvents. In some embodiments, the aqueous solution of apixatan comprises HPBCD and/or povidone as a solubility enhancing agent. In some embodiments, the aqueous solution of apixaban optionally comprises a sweetening agent and/or a flavoring agent.

In some embodiments, the aqueous solution of apixaban comprises less than 35% w/v propylene glycol based on total weight/volume of the formulation. In some embodiments, the aqueous solution of apixaban comprises maximum up to 400 mg glycerin based on total weight/volume of the formulation. In some embodiments, the aqueous solution of apixaban comprises added povidone, and propylene glycol.

Thus, in a preferred embodiment, the present invention provides an aqueous solution of apixaban consisting essentially of apixaban and a cyclodextrin, or a derivative thereof wherein the said aqueous solution is devoid of any surfactants. In another preferred embodiment, the present invention provides an aqueous solution of apixaban consisting essentially of apixaban and a polymer wherein the said aqueous solution is devoid of any surfactants.

In another preferred embodiments, the present invention provides an aqueous solution of apixaban consisting of apixaban, a cyclodextrin, or a derivative thereof, a preservative, a solvent, a pH adjusting agent or a buffering agent, a vehicle and optionally a flavor and/or a sweetener.

In another preferred embodiments, the present invention provides an aqueous solution of apixaban consisting of apixaban, polyvinylpyrrolidone (povidone), a preservative, a solvent, a pH adjusting agent or a buffering agent, a vehicle and optionally a flavor and/or a sweetener.

In some embodiments, level of the 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxylic acid impurity in the aqueous oral solutions of apixaban of the present invention is less than about 1.0% w/w, preferably less than about 0.5% w/w, and more preferably less than about 0.3% w/w. In some of the preferred embodiments of the present invention, level of the 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxylic acid impurity in the aqueous oral solutions of apixaban of the present invention is less than about 0.1% w/w or less than about 0.05% w/w.

In some embodiments, the total impurities present in the apixaban aqueous oral solution of the present invention are less than about 5% w/w or less than about 3% w/w or less than about 1% w/w or less than about 0.5% w/w.

### Excipients of the Present Invention

Vehicles are the liquid bases that carry drugs and other excipients in dissolved or dispersed state. Vehicles may be aqueous or non-aqueous or mixture thereof. Non-aqueous solvents/co-solvents may also be added to the formulations of the present invention to increase the solubility of poorly soluble substances and enhance the chemical stability of a drug. Suitable solvents/co-solvents, and vehicles that may be employed, in the apixaban formulations of the invention include, but are not limited to, dichloromethane, acetonitrile, ethyl acetate, acetone, propylene carbonate, glycerin, coconut fatty acid diethanolamide, medium and/or long chain fatty acids or glycerides, monoglycerides, diglycerides, triglycerides, structured triglycerides, soyabean oil, peanut oil, corn oil, corn oil monoglycerides, corn oil diglycerides, corn oil triglycerides, polyethylene glycol, caprylocaproylmacroglycerides, caproyl 90, propylene glycol, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, acetone, methyl isobutyl ketone, methyl ethyl ketone and the like or any combinations thereof.

The pH of an oral liquid formulation is a key point in many regards. Control of the formulation pH, could prevent large changes during storage. Therefore, most formulations utilize a buffer to control potential changes in the solution pH. The amount of buffer capacity needed is generally between 0.01 and 0.1 M, and a concentration between 0.05 and 0.5 M is usually sufficient. The selection of a suitable buffer should be based on (i) Whether the acid-base forms are listed for use in oral formulations, (ii) The stability of the drug and excipients in the buffer, and (iii) The compatibility between the buffer and container. A combination of buffers can also be used to gain a wider range of pH compared to the individual buffer alone. However, not all buffers are suitable for use. For example, a boric acid buffer may be used for optical and IV delivery but not in oral liquids because of its toxicity. Non-limiting examples of buffers include inorganic acids, organic acids, salts of inorganic & organic acids, aminoacids, inorganic bases and organic bases such as acetic acid, adipic acid, ammonium carbonate, ammonium hydroxide, ammonium phosphate, boric acid, citric acid, diethanolamine, fumaric acid, hydrochloric acid, malic acid, nitric acid, propionic acid, potassium acetate, potassium bicarbonate, potassium chloride, potassium citrate, potassium metaphosphate, potassium phosphate, sodium acetate, sodium bicarbonate, sodium borate, sodium carbonate, sodium chloride, sodium citrate, sodium glycolate, sodium hydroxide, sodium lactate, sodium phosphate, sodium proprionate, succinic acid, oxalic acid, sulfuric acid, tartaric acid, triethylamine, triethanolamine, tromethamine, trolamine and the like or combinations thereof. The concentration of pH adjusting agent/buffer in the present invention ranges from 0.001% to 10%, based on total weight/volume of the formulation.

A “buffering agent” or “buffer” as used herein is a system that is used for the purposes and is capable of maintaining the desired/required pH of the formulations throughout desired/required time period, e.g. stability studies and/or shelf life of the drug product. The desired pH of the liquid formulation according to the present invention is between about 3.0 and about 7.0, however, a preferred pH is between about 3.0 and about 6.0 and a particularly preferred pH is about 4.0.

Microbiological contamination presents a significant health hazard in oral liquids. Therefore, the use of preservatives become inevitable to prevent the growth of microorganisms during the product's manufacture and shelf life, although it may be most desirable to develop a “preservative-free” formulation to address the increasing concerns about the biological activity of these compounds. Most formulations require some kind of preservative to ensure no microbial growth.

The majority of preservatives are bacteriostatic rather than bacteriocidal, and consists of both acid and nonacid types. Among the acidic types are phenol, chloro-cresol, 9-phenyl phenol, alkyl esters of para-hydroxybenzoic acid, benzoic acid, boric acid, and sorbic acid, and their respective salts. Therefore, the pH of solution, and the pKa of the preservative need to be carefully evaluated prior to selecting a preservative for a formulation. Neutral preservatives include chlorobutanol, benzyl alcohol, and beta-phenylethyl alcohol. Under alkaline conditions, it is generally regarded that microbial growth is insignificant and at these pH values, the need for a preservative is not generally recommended.

Many preservatives listed in the FDA inactive ingredient guide for liquid & solid dosage forms. Unfortunately, many of them are not recommended for use in oral liquids and hence the choice of an acceptable preservative for an oral liquid formulation is limited. In addition, the solubility of many preservatives in aqueous system may not be high enough for effective antimicrobial activity. Additionally, it is essential to understand that bacteriostatic agents like para hydroxyl benzoic acids can partition between organic and aqueous phases in a heterogenous liquid formulations in such a way that their activity is significantly reduced. Non-limiting examples of preservatives include alcohol, ethanol, chlorobutanol, phenoxyethanol, potassium benzoate, benzyl alcohol, benzoic acid, potassium sorbate, sorbic acid, benzalkonium chloride, benzethonium chloride, cetrimonium bromide, cetylpyridinium chloride, bronopol, chlorbutol, chlorocresol, cresol, butylparaben or its salts such as sodium, methylparaben or its salts such as sodium, propylparaben or its salts such as sodium, ethylparaben or its salts such as sodium, phenol, thymol, phenylethanol, sodium benzoate, and the like. The concentration of a preservative in the present invention ranges from 0.001% to 10%, based on total weight/volume of the formulation.

Sweetening agents when used in the formulations of the present invention, without limitation include, Glucose, Sucralose, Trehalose, Fructose, Xylose, Dextrose, Galactose, Tagatose, Maltose, Sucrose, Glycerol, Dulcitol, Mannitol, Lactitol, Sorbitol, Xylitol, Saccharine or the corresponding sodium, potassium or calcium salt, Cyclamate or the corresponding sodium or calcium salt, Aspartame, or Acesulfame or the potassium salt thereof, Dulcin or Ammonium glycyrrhizinate, Neotame, Alitame, Inulin, Isomalt, Neohesperidin dihydrochalcone, Thaumatin and the like or any combinations thereof. The concentration of sweetener ranges from 0.001% to 10%, based on total weight/volume of the formulation.

Flavoring agents when used in the formulations of the present invention, without limitation include, synthetic flavor oils and flavoring aromatics and/or natural oils, extracts from plants leaves, flowers, fruits, and so forth and the like or any combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth and the like or any combinations thereof. Solid forms, such as spray dried forms of flavoring agents, may also be useful in the liquid formulations of apixaban disclosed herein. The concentration of flavor ranges from 0.001% to 10%, based on total weight/volume of the formulation.

The solvent/co-solvent according to the present invention is used in a quantity sufficient which helps dissolve apixaban in the formulation. The vehicle according to the present invention is used in a quantity sufficient to bring the final volume of the apixaban formulation to the desired volume (e.g. to the total batch size). The sweetening agent and/or flavoring agent when used in the apixaban formulation are used in a quantity sufficient to make the formulation of the present invention palatable.

An apixaban formulation according to any of the embodiments of the present invention wherein the formulation may further comprises antioxidants. Antioxidants are compounds that can reduce a drug that has been oxidized, or compounds that are more readily oxidized than the agents they are to protect (oxygen scavengers).

Some substances prone to oxidation include unsaturated oils/fats, compounds with aldehyde or phenolic groups, colors, flavors, sweeteners, plastics and rubbers, the latter being used in containers for products. Oxidation may manifest as products with an unpleasant odour, taste, appearance, precipitation, discoloration or even a slight loss of activity. The term rancidity refers to many typical off-flavors that result from autoxidation of unsaturated fatty acids that are present in oils and fats, and it affects many oils and fats. The distinct rancid odour may result from short-chain, volatile monomers resulting from the cleavage of the longer chain, less volatile oils and fats. Non-limiting examples of anti-oxidants are α-Tocopherol acetate, Ascorbic acid, Erythorbic acid, Butylated hydroxytoluene (BHT), d-α-Tocopherol natural, Monothioglycerol, Sodium bisulfite, Sodium sulfite, Sodium metabisulfite, Potassium metabisulfite, Acetone sodium bisulfite, Ascorbyl palmitate, Cysteine, d-α-tocopherol synthetic, Nordihydroguaiaretic acid, Sodium formaldehyde sulfoxylate, Sodium thiosulfate, Acetylcysteine, Ascorbyl palmitate, Butylated hydroxyanisole (BHA), Cysteine hydrochloride, Dithiothreitol, Propyl gallate, Thiourea and the like or any combinations thereof. The concentration of an antioxidant in the present invention ranges from 0.001% to 10%, based on total weight/volume of the formulation.

### Active Pharmaceutical Ingredient and its Amount Used in the Present Invention

In accordance with the present invention, the term “apixaban” unless indicated otherwise in the specification refers to apixaban in the form of free base or its pharmaceutically acceptable salt, amorphous, crystalline or any isomer or derivative, hydrate or solvate, prodrug or combinations thereof. Preferably, apixaban is in the form of its free base.

The apixaban formulation of the present invention may comprise from about 0.50 mg/mL to about 5.0 mg/mL of apixaban. This formulation may include, for example, about 0.5 mg of apixaban, about 0.75 mg of apixaban, about 1.00 mg of apixaban, about 1.25 mg of apixaban, about 1.50 mg of apixaban, about 1.75 mg of apixaban, about 2.00 mg of apixaban, about 2.25 mg of apixaban, about 2.50 mg of apixaban, about 2.75 mg of apixaban, about 3.00 mg of apixaban, about 3.25 mg of apixaban, about 3.50 mg of apixaban, about 3.75 mg of apixaban, about 4.00 mg of apixaban, about 4.25 mg of apixaban, about 4.50 mg of apixaban, about 4.75 mg of apixaban, and about 5.00 mg of apixaban, or any amount of apixaban in between these values. In alternative embodiments, the apixaban formulation of the present invention may comprise from about 0.25 mg/mL to about 10 mg/mL apixaban or any amount of apixaban in between these values. In alternative embodiments, the apixaban formulation of the present invention may comprise from about 0.5 mg/mL to about 10 mg/mL apixaban or any amount of apixaban in between these values.

Apixaban has known as well as unknown impurities. In particular, O-desmethoxy impurity, amino acid impurity, acid impurity, chloro impurity, ester impurity and methyl ester impurity were monitored.

### Potential Impurities for Apixaban

| Sr | Name | Type | Structure | Chemical name |
|---|---|---|---|---|
| 1 | Acid impurity | Process/ degradation impurity | 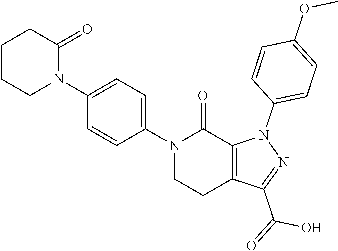 [SMILES: COc1ccc(-n2nc(C(=O)O)c3c2C(=O)N(c2ccc(N4CCCCC4=O)cc2)CC3)cc1] | 1-(4-methoxyphenyl)-7- oxo-6-(4-(2-oxopiperidin- 1-yl)phenyl)-4,5,6,7- tetrahydro-1H- pyrazolo[3,4-c]pyridine-3- carboxylic acid |
| 2 | Amino acid impurity | Process/ degradation impurity | 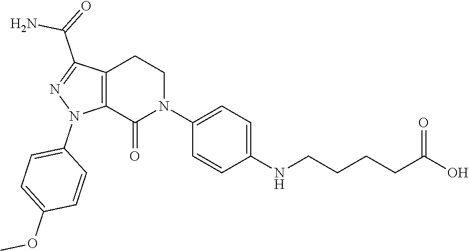 [SMILES: COc1ccc(-n2nc(C(N)=O)c3c2C(=O)N(c2ccc(NCCCCC(=O)O)cc2)CC3)cc1] | 5-((4-(3-carbamoyl-1-(4- methoxyphenyl)-7-oxo- 1,4,5,7-tetrahydro-6H- pyrazolo[3,4-c]pyridin-6- yl)phenyl)amino)pentanoic acid |
| 3 | Dehydro impurity | Process/ degradation impurity | 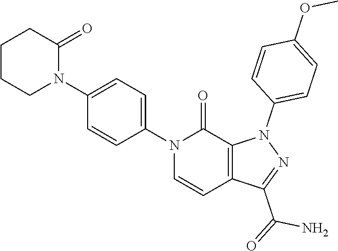 [SMILES: COc1ccc(-n2nc(C(N)=O)c3ccn(-c4ccc(N5CCCCC5=O)cc4)c(=O)c32)cc1] | 1-(4-methoxyphenyl)-7- oxo-6-(4-(2-oxopiperidin- 1-yl)phenyl)-6,7-dihydro- 1H-pyrazolo[3,4- c]pyridine-3-carboxamide |
| 4 | Chloro impurity | Process impurity | 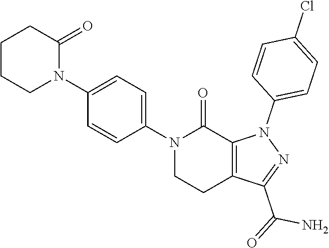 [SMILES: NC(=O)c1nn(-c2ccc(Cl)cc2)c2c1CCN(c1ccc(N3CCCCC3=O)cc1)C2=O] | 1-(4-chlorophenyl)-7-oxo- 6-(4-(2-oxopiperidin-1- yl)phenyl)-4,5,6,7- tetrahydro-1H- pyrazolo[3,4-c]pyridine-3- carboxamide |
| 5 | Methyl ester impurity | Process impurity | 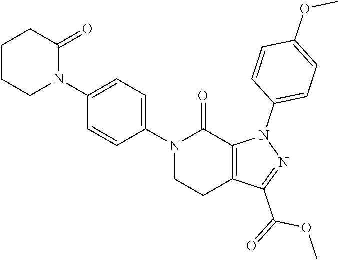 [SMILES: COC(=O)c1nn(-c2ccc(OC)cc2)c2c1CCN(c1ccc(N3CCCCC3=O)cc1)C2=O] | Methyl 1-(4- methoxyphenyl)-7-oxo-6- (4-(2-oxopiperidin-1- yl)phenyl)-4,5,6,7- tetrahydro-1H- pyrazolo[3,4-c]pyridine-3- carboxylate |
| 6 | Ester impurity | Process impurity | 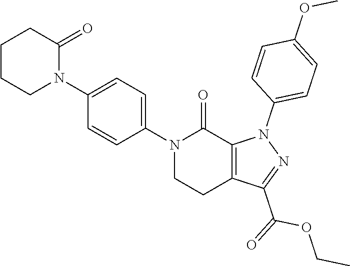 [SMILES: CCOC(=O)c1nn(-c2ccc(OC)cc2)c2c1CCN(c1ccc(N3CCCCC3=O)cc1)C2=O] | Ethyl 1-(4- methoxyphenyl)-7-oxo-6- (4-(2-oxopiperidin-1- yl)phenyl)-4,5,6,7- tetrahydro-1H- pyrazolo[3,4-c]pyridine-3- carboxylate |

### Process of Preparation of Apixaban Formulations of the Present Invention

In a yet another embodiment, the present invention provides a process of preparing the non-aqueous oral solution of apixaban, which comprises following steps:
1. Desired quantity of apixaban is dissolved in required quantity of Labrasol® under heating at 70-80° C.;
2. Optionally sweetening agent is dissolved in the solution of step-1 under heating at 70-80° C.;
3. After cooling down the solution of step-2 to room temperature, required quantity of Phosal® 50 PG is added;
4. Optionally flavoring agent is added to the solution of step-3;
5. Optionally viscosity modifying agent is added to the solution of step-4; and
6. Required quantity of medium chain triglyceride is added to bring the final volume of the formulation to the desired volume.
7. The bulk solution is filtered and packaged in the amber glass bottle (preferably 150 mL) with child resistant closure.

In a yet another embodiment, the present invention provides a process of preparing the aqueous oral solution of apixaban, which comprises following steps:
1. Required quantities of glycerin, propylene glycol and polyethylene glycol are mixed and a solubility enhancer (a cyclodextrin, or a derivative thereof or a polymer) is added into it;
2. Required amount of water is added to the mixture of step-1 and heated to about 40° C. to form a clear solution;
3. A pH adjusting agent or a buffering agent is added to the solution of step-2 to adjust the pH of the solution about 4.0;
4. Required amount of apixaban is added into the solution of step-3 and dissolved under hearing at about 40° C. and stirring;
5. A preservative is added in a require amount and dissolved into the solution of step-4 under heating at about 40° C.; and
6. Required quantity of water is added to the solution of step-5 to make up the volume to the final batch size and the solution is cooled down to room temperature.

A skilled person knows that some variations can be made to the above-mentioned process and order of addition of different ingredients can be changed without diminishing the quality of the product. All such variations are within the ambit of the present invention. Ethanol may also be used in the process of the preparation of the aqueous solution of the present invention, however, the finished product or end formulation (aqueous formulation) does not contain ethanol.

### Routes/Methods of Administration of the Apixaban Formulation of the Present Invention

The apixaban formulation of the present invention can be administered orally by conventional means such as swallowing or can be administered through feeding tubes. Feeding tubes can be inserted via a number of routes: via the nasopharynx, for example nasogastric (NG) or nasojejunal (NJ), or via direct access to the GI tract through the skin, for example gastrostomy or jejunostomy tubes. The invention is suitable for use through nasogastric or other feeding tubes also.

The apixaban formulation of the present invention is adjustable to the prescribed dosing scheme and can be administrated flexibly according to the desired pharmaceutical dosing scheme. The dose volume of the apixaban formulation of the present invention can be multiplied or divided. Therefore, patient compliance is achieved and swallowing is facilitated. It can be administrated in multiple times during the day.

### Use/Method of Use/Therapeutic Efficacy of the Apixaban Formulation of the Present Invention

The apixaban formulation of the present invention provides similar bioavailability and pharmacokinetic properties to ELIQUIS® (apixaban) tablets. For instance, the apixaban formulation of the present invention has a Cₘₐₓ, AUC_{inf}, and/or AUC_{(0-T)} from 80% to 125% of the Cₘₐₓ, AUC_{inf}, and/or AUC_{(0-T)}, respectively, of an apixaban oral tablet comprising crystalline apixaban particles having a D₉₀ (90% of the volume) as measured by laser light scattering, equal to or less than about 89 μm, and a pharmaceutically acceptable diluent or carrier, as described in the U.S. Pat. No. 9,326,945 assigned to the Bristol Myers & Squibb Company and Pfizer Inc. Such diluent or carrier may include anhydrous lactose, microcrystalline cellulose, croscarmellose sodium, magnesium stearate, sodium lauryl sulfate, opadry dispersion, or any combination thereof.

In a yet another embodiment, the present invention provides a method for treating a thromboembolic disorder comprising administering to a patient in need thereof a therapeutically effective amount of an apixaban formulation of the present invention.

In a yet another embodiment, the present invention provides an apixaban formulation of the present invention for use in treating a thromboembolic disorder.

In a yet another embodiment, the present invention provides use of an apixaban formulation of the present invention in the treatment of a thromboembolic disorder.

In yet another embodiment, the present invention provides use of an apixaban formulation of the present invention in the preparation of a medicament for use in treating a thromboembolic disorder.

Thromboembolic disorders mentioned in the foregoing paragraphs include those disclosed in the U.S. Pat. No. 6,967,208 assigned to Bristol Myers and Squibb Company. Non-limiting examples of thromboembolic disorders are arterial cardiovascular thromboembolic disorders, venous cardiovascular thromboembolic disorders, and thromboembolic disorders in the chambers of the heart. Thromboembolic disorders may also include unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

### Kits/Packaging Materials of the Apixaban Formulation of the Present Invention

In some embodiments, the present invention provides a kit of an apixaban formulation of the present invention for oral administration. In some embodiments, the pharmaceutical kit comprises a container comprising the liquid formulation according to any one of the embodiments, (ii) optionally, a calibrated device, and optionally (iii) instructions for administration. Preferably, the container is of glass and/or plastic material and more preferably is an amber glass bottle. In some embodiments, the container is a multi-use container. In some embodiments, the container is a single-use container. Preferably, in some embodiments, the calibrated device is selected from an oral syringe, a cup, a dropper, or a spoon. In a preferred embodiment, the glass and plastic bottles will be available in 30, 60, 100, 120, 150, 250 & 500 mL fill volumes.

### Definitions

Unless defined otherwise, all the technical and scientific terms used herein have the same meanings as commonly known by a person skilled in the art. In case of conflict, the definitions provided herein will prevail.

The term “essentially non-aqueous” within the scope of the invention is to be understood that the formulation is substantially free of water. Alternatively, the formulation of the present invention does not contain any added water. The term “essentially aqueous” within the scope of the invention is to be understood that the formulation contains substantial amount of added water.

Within the context of this invention, the term “solution” refers to a mixture of one or more substances dispersed molecularly (i.e., dissolved) in a dissolving liquid medium or vehicle. The solution is preferably homogeneous, in the sense that Active Pharmaceutical Ingredient (API) is essentially uniformly dissolved and distributed in the solution.

As used herein, the term “cyclodextrin, or a derivative thereof” can refer to a cyclic dextrin molecule that is formed by enzyme conversion of starch. Specific enzymes, e.g., various forms of cycloglycosyltransferase (CGTase), can break down helical structures that occur in starch to form specific cyclodextrin molecules having three-dimensional polyglucose rings with, e.g., 6, 7, or 8 glucose molecules. For example, α-CGTase can convert starch to α-cyclodextrin having 6 glucose units, β-CGTase can convert starch to β-cyclodextrin having 7 glucose units, and γ-CGTase can convert starch to γ-cyclodextrin having 8 glucose units. Cyclodextrins include, but are not limited to, at least one of α-cyclodextrin, β-cyclodextrin, γ-cyclodextrin, and combinations thereof. β-cyclodextrin is not known to have any toxic effects, is World-Wide GRAS (i.e., Generally Regarded As Safe) and natural, and is FDA approved. α-cyclodextrin and γ-cyclodextrin are also considered natural products and are U.S and E.U. GRAS.

The term “beta-cyclodextrin” as used herein refers to cyclic alpha-1, 4-linked oligosaccharides of a D-glucopyranose containing a relatively hydrophobic central cavity and hydrophilic outer surface. Particular usefulness has been observed in the present invention utilizing hydroxypropyl-beta-cyclodextrin (HPBCD) or sulfobutylether-beta-cyclodextrin (SBECD), however, other substituted and unsubstituted beta-cyclodextrins can also be used in the practice of the invention. Non-limiting examples of cyclodextrin derivative include hydroxypropyl-γ-cyclodextrin, randomly methylated β-cyclodextrin, carboxymethyl-β-cyclodextrin or sulfobutyl-β-cyclodextrin.

As used herein, the term “about” is synonymous with “approximately” and is used to provide flexibility to a numerical value or range endpoint by providing that a given value may be “a little above” or “a little below” the value stated. “About” can mean within a percentage range of a given value. For example, the range can be ±1%, ±5%, ±10%, ±20%, ±30%, ±40% or ±50% of a given value. However, it is to be understood that even when a numerical value is accompanied by the term “about” in this specification, that express support shall be provided at least for the exact numerical value as well as though the term “about” were not present.

The use of the terms “a” and “an” and “the” and similar referents in the context of describing the invention (especially in the context of the claims) are to be construed to cover both the singular and the plural, unless otherwise indicated herein or clearly contradicted by context.

The expression “% w/w” as used herein, refers to the percent weight of an identified component based on the total weight of the liquid formulation. The expression “% w/v” as used herein, refers to the percent weight of an identified component based on the total volume of the liquid formulation.

The terms “related substances”, “impurities” and “degradation products” as used herein are synonyms of each other and can be used interchangeably, which represent the same meaning.

As used herein, “optional” or “optionally” means that the subsequently described event or circumstance does or does not occur, and that the description includes instances where said event or circumstance occurs and instances where it does not. For example, an optionally substituted group means that the group is un-substituted or is substituted.

As used herein, “comprises”, “comprising”, “containing” and “having” and the like can have the meaning ascribed to them in patent law and can mean “includes”, “including” and the like, and are generally interpreted to be open ended terms. The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility). The terms “consisting of” or “consists of” are closed terms, and include only the components, structures, steps, or the like specifically listed in conjunction with such terms, as well as that which is in accordance with patent law. “Consisting essentially of” or “consists essentially of” have the meaning generally ascribed to them by patent law. In particular, such terms are generally closed terms, with the exception of allowing inclusion of additional items, materials, components, steps, or elements, that do not materially affect the basic and novel characteristics or function of the item(s) used in connection therewith.

The term “pharmaceutically acceptable” as used herein refers to an ingredient having compatibility with the other ingredients of the formulation and not deleterious to the recipient thereof.

The term “pharmaceutically acceptable salt” refers to salts of free base or free acid which are formed with inorganic or organic acids/bases.

The term “quantity sufficient” or “q.s.” as used herein refers to the amount of a substance required to meet a certain parameter.

Amounts of apixaban and other components identified here were determined chromatographically, e.g., High Performance Liquid Chromatograph (HPLC). Amounts of apixaban related substances identified here were also determined chromatographically, e.g., High Performance Liquid Chromatograph (HPLC).

The term “stable” means that no more than 10% loss of apixaban under typical commercial storage conditions. Preferably, formulations of the present invention will have no more than about 8% loss of apixaban, more preferably, no more than about a 5% loss of apixaban, more preferably, no more than about a 3% loss of apixaban, and most preferably, no more than about a 2% loss of apixaban under typical commercial storage conditions (i.e., at 25° C./60% Relative Humidity or at 40° C./75% Relative Humidity) for at least 2 months, at least 3 months, at least 6 months, at least 9 months, at least 12 months, at least 15 months, at least 18 months, at least 21 months, at least 24 months, at least 30 months or at least 36 months.

As used herein, “treatment” refers to ameliorating or reducing symptoms associated with a disease or condition. Treatment means any manner in which the symptoms of a condition, disorder or disease are ameliorated or otherwise beneficially altered. Hence, treatment encompasses prophylaxis, therapy and/or cure. Treatment also encompasses any pharmaceutical use of the compositions herein.

As used herein an “effective amount” of a compound or composition for treating a particular disease is an amount that is sufficient to ameliorate, or in some manner reduce symptoms to achieve the desired physiological effect. Such amount can be administered as a single dosage or can be administered according to a regimen, whereby it is effective. The effective amount is readily determined by one of skill in the art following routine procedures.

As used herein, “disease” or “disorder” or “condition” refers to a pathological condition in an organism resulting from cause or condition including, but not limited to, infections, acquired conditions, genetic conditions, and characterized by identifiable symptoms.

As used herein, “patient” or “subject” to be treated includes humans and or non-human animals, including mammals. Mammals include primates, such as humans, chimpanzees, gorillas and monkeys; domesticated animals, such as dogs, horses, cats, pigs, goats, cows; and rodents such as mice, rats, hamsters and gerbils.

### BEST MODE OF CARRYING OUT THE INVENTION

### Example(s)

The following examples are exemplary and not intended to be limiting. The above disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

### Example-1: Preparation of the Non-Aqueous Solution of Apixaban (without Viscosity Modifying Agent)

| S. | | Quantity | | Functional |
|---|---|---|---|---|
| No. | Ingredients | mg/mL | % w/v | Category |
| 1 | Apixaban | 1.00 | 0.1 | Active |
| | | | | Pharmaceutical |
| | | | | Ingredient |
| 2 | Caprylocaproyl | 200.0 | 20 | Solvent |
| | Polyoxyl-8 glycerides | | | |
| | (Labrasol ®) | | | |
| 3 | Neotame | 0.10 | 0.01 | Sweetener |
| 4 | Orange flavour | 2.00 | 0.2 | Flavour |
| 5 | Phosal ® 50 PG | 600.0 | 60 | Vehicle |
| 6 | Medium chain | Q.S. to 1 mL | Q.S. to 100% | Vehicle |
| | triglycerides | | | |

Q.S. = quantity sufficient

Surprisingly, an apixaban solution of the present invention comprising apixaban at concentrations within the range of 0.25 mg/mL to 10 mg/mL and less than 35% (w/v) propylene glycol based on the total formulation, dissolves apixaban in small vehicle and solvent/co-solvent volumes, resulting in a solution which remains stable and is free of precipitation/crystallization.

TABLE 1
Stability study results of a non-aqueous solution of apixaban
of Example-1

| Test/ | | 25° C. | | 40° C. | |
|---|---|---|---|---|---|
| Parameter | Initial | 1 month | 2 months | 1 month | 2 months |
| Des- | Yellow | Yellow | Yellow | Yellow | Yellow |
| cription | Colored | colored | colored | colored | colored |
| | solution | solution | solution | solution | solution |
| % Assay | 96.6 | 96.2 | 96.1 | 96.5 | 95.5 |
| of | | | | | |
| apixaban | | | | | |

Related substances (% w/w)

| | | | | | |
|---|---|---|---|---|---|
| Acid | 0.01 | 0.02 | 0.02 | 0.02 | 0.02 |
| impurity | | | | | |
| Amino | 0.02 | 0.02 | 0.04 | 0.02 | 0.08 |
| impurity | | | | | |
| Dehydro | 0.02 | 0.03 | 0.04 | 0.07 | 0.07 |
| impurity | | | | | |
| Any | — | — | — | — | 0.03 |
| individual | (RRT-0.43) | (RRT-0.43) | (RRT-0.43) | (RRT- | (RRT- |
| un- | | | | 0.43) | 0.43) |
| specified | 0.16 | 0.16 | 0.15 | 0.13 | 0.13 |
| impurity | (RRT-0.78) | (RRT-0.78) | (RRT-0.78) | (RRT- | (RRT- |
| | | | | 0.78) | 0.78) |
| | 0.13 | 0.09 | 0.21 | 0.18 | 0.20 |
| | (RRT-0.85) | (RRT-0.85) | (RRT-0.85) | (RRT- | (RRT- |
| | | | | 0.85) | 0.85) |
| | — | 0.01 | 0.04 | — | — |
| | (RRT-0.93) | (RRT-0.93) | (RRT-0.93) | (RRT- | (RRT- |
| | | | | 0.93) | 0.93) |
| | — | — | — | — | 0.07 |
| | (RRT-1.27) | (RRT-1.27) | (RRT-1.27) | (RRT- | (RRT- |
| | | | | 1.27) | 1.27) |
| | — | — | — | — | 0.06 |
| | (RRT-1.43) | (RRT-1.43) | (RRT-1.43) | (RRT- | (RRT- |
| | | | | 1.43) | 1.43) |
| Total | 0.34 | 0.33 | 0.50 | 0.42 | 0.66 |
| impurities | | | | | |

### Example-2: Preparation of the Non-Aqueous Solution of Apixaban (without Viscosity Modifying Agent)

| S. | | Quantity | | Functional |
|---|---|---|---|---|
| No. | Ingredients | mg/mL | % w/v | Category |
| 1 | Apixaban # | 1.0 | 0.1 | Active |
| | | | | Pharmaceutical |
| | | | | Ingredient |
| 2 | Caprylocaproyl | 180.0 | 18 | Solvent |
| | Polyoxy1-8 glycerides | | | |
| | (Labrasol ®) | | | |
| 3 | Neotame | 1.0 | 0.1 | Sweetener |
| 4 | Orange flavour | 2.0 | 0.2 | Flavour |
| 5 | Phosal ® 50 PG | 600.0 | 60 | Vehicle |
| 6 | Medium chain | Q.S. to 1 mL | Q.S. to 100% | Vehicle |
| | triglycerides | | | |

TABLE 2
Stability study results of a non-aqueous solution of apixaban of Example-2

| Test/ | | 25° C./60% RH | | 30° C./65% RH | | 40° C./75% RH | | |
|---|---|---|---|---|---|---|---|---|
| Parameter | Initial | 3M | 6M | 3M | 6M | 1M | 3M | 6M |
| Description | Light | Light Yellow | Light Yellow | Light Yellow | Light | Light | Light | Light |
| | Yellow | colored | colored | colored | Yellow | Yellow | Yellow | Yellow |
| | colored | solution | solution | solution | colored | colored | colored | colored |
| | solution | | | | solution | solution | solution | solution |
| % Assay | 99.6 | 100.4 | 99.1 | 99.8 | 100.3 | 99.2 | 99.7 | 98.8 |
| of | | | | | | | | |
| apixaban | | | | | | | | |

Related substances (% w/w)

| | | | | | | | | |
|---|---|---|---|---|---|---|---|---|
| Acid | 0.03 | 0.03 | 0.03 | 0.03 | 0.03 | 0.03 | 0.03 | 0.03 |
| impurity | | | | | | | | |
| Any | 0.02 | 0.01 | 0.05 | 0.01 | 0.06 | 0.02 | 0.05 | 0.17 |
| Individual | | | | | | | | |
| unspecified | | | | | | | | |
| impurity | | | | | | | | |
| Total | 0.05 | 0.04 | 0.16 | 0.04 | 0.17 | 0.06 | 0.09 | 0.24 |
| impurities | | | | | | | | |

### Example-3: Drug-Excipients Compatibility Study

The drug excipient compatibility study is an important part of the formulation development to understand the role of the inactive ingredients intended to be used on the product quality. The thermal stability study was carried out to check the compatibility of each inactive ingredient with the active ingredient.

TABLE 3
Formulation details of Drug Excipient compatibility study

| | | | API: Excipient |
|---|---|---|---|
| S. No. | Batch No. | Composition | Ratio |
| 1 | APAL1007A | Apixaban | 10 mg |
| 2 | APAL1007B | Apixaban + Phosal PG 50 | 10 mg: 1 gm |
| 3 | APAL1007C | Apixaban + Medium chain | 10 mg: 10 gm |
| | | triglycerides (MCT) | |
| 4 | APAL1007D | Apixaban + ethanol | 10 mg: 790 mg |
| 5 | APAL1007E | Apixaban + Labrasol ® | 10 mg: 6.0 gm |
| 6 | APAL1007F | Apixaban + sweetening agent | 10 mg: 2 mg |
| | | (Neotame) | |
| 7 | APAL1007G | Apixaban + Silicon dioxide | 10 mg: 260 mg |
| 8 | APAL1007H | Apixaban + Flavor (Orange) | 10 mg: 40 mg |
| 9 | APAL1007I | Apixaban + Phosal PG 50 + | 10 mg + 1 gm + |
| | | MCT + ethanol + Labrasol + | 4 gm + 800 mg + |
| | | Neotame + Orange | 4 gm + 2 mg + |
| | | Flavor + Silicon dioxide | 40 mg + 150 mg |

The results for the Drug-excipients compatibility study are mentioned below:

TABLE 4
Stability study results of API at various storage conditions

| Batch No. | APAL1007A | | | | | |
|---|---|---|---|---|---|---|
| | | 15 | 30 | 30 | 30 | 30 |
| | | Days | Days | Days | Days | Days |
| | | 60° C. | 25° C. | 25° C. | 40° C. | 40° C. |
| Stability | | Close | Close | Open | Open | Close |
| time point | Initial | Vial | Vial | Vial | Vial | Vial |
| Description | White | White | White | White | White | White |
| | color | color | color | color | color | color |
| | powder | powder | powder | powder | powder | powder |

Related substances (% w/w)

| | | | | | | |
|---|---|---|---|---|---|---|
| Acid | 0.01 | 0.02 | 0.01 | 0.02 | 0.02 | 0.02 |
| Impurity | | | | | | |
| Any | 0.02 | 0.03 | 0.03 | 0.02 | 0.03 | 0.03 |
| individual | | | | | | |
| unspecified | | | | | | |
| impurity | | | | | | |
| Total | 0.06 | 0.07 | 0.07 | 0.07 | 0.09 | 0.09 |
| impurities | | | | | | |

NMT = not more than

TABLE 5
Stability study results of API with Phosal 50 PG at various storage
conditions

| Batch No. | APAL1007B | | | | | |
|---|---|---|---|---|---|---|
| | | 15 Days | 30 Days | 30 Days | 30 Days | 30 Days |
| | | 60° C. | 25° C. | 25° C. | 40° C. | 40° C. |
| Stability | | Close | Close | Open | Open | Close |
| time point | Initial | Vial | Vial | Vial | Vial | Vial |
| Description | Yellow | Yellow | Yellow | Yellow | Yellow | Yellow |
| | color | color | color | color | color | color |
| | solution with | solution with | solution with | solution with | solution with | solution with |
| | suspended | suspended | suspended | suspended | suspended | suspended |
| | particle | particle | particle | particle | particle | particle |

Related Substances (% w/w)

| | | | | | | |
|---|---|---|---|---|---|---|
| Acid Impurity | 0.02 | 0.02 | 0.02 | 0.02 | 0.02 | 0.02 |
| Any individual | 0.02 | 0.06 | 0.02 | 0.02 | 0.04 | 0.03 |
| unspecified | | | | | | |
| impurity | | | | | | |
| Total impurities | 0.05 | 0.19 | 0.07 | 0.05 | 0.16 | 0.08 |

TABLE 6
Analytical results API with MCT at various storage condition

| Batch No. | APAL1007C | | | | | |
|---|---|---|---|---|---|---|
| | | 15 Days | 30 Days | 30 Days | 30 Days | 30 Days |
| Stability | | 60° C. | 25° C. | 25° C. | 40° C. | 40° C. |
| time | | Close | Close | Open | Open | Close |
| point | Initial | Vial | Vial | Vial | Vial | Vial |
| Description | Off | Off | Off | Off | Off | Off |
| | white | white | white | white | white | white |
| | Turbid | turbid | turbid | turbid | turbid | turbid |
| | solution | solution | solution | solution | solution | solution |

Related Substances (% w/w)

| | | | | | | |
|---|---|---|---|---|---|---|
| Acid Impurity | 0.02 | 0.02 | 0.02 | 0.02 | 0.03 | 0.03 |
| Any individual | 0.02 | 0.03 | 0.02 | 0.02 | 0.03 | 0.02 |
| unspecified | | | | | | |
| impurity | | | | | | |
| Total | 0.08 | 0.07 | 0.07 | 0.07 | 0.10 | 0.09 |
| impurities | | | | | | |

TABLE 7
Stability study results of API with ethanol at various
storage conditions

| Batch No. | APAL1007D | | | | | |
|---|---|---|---|---|---|---|
| | | 15 | 30 | 30 | 30 | 30 |
| | | Days | Days | Days | Days | Days |
| Stability | | 60° C. | 25° C. | 25° C. | 40° C. | 40° C. |
| time | | Close | Close | Open | Open | Close |
| point | Initial | Vial | Vial | Vial | Vial | Vial |
| Description | Off | Colorless | Off | Off | Off | Off |
| | white | solution | white | white | white | white |
| | turbid | Containing | turbid | powder | powder | turbid |
| | solution | crystals/ | solution | | | solu- |
| | | particles. | | | | tion |

Related Substances (% w/w)

| | | | | | | |
|---|---|---|---|---|---|---|
| Acid | 0.02 | 0.02 | 0.02 | 0.02 | 0.02 | 0.02 |
| Impurity | | | | | | |
| Any | 0.01 | 0.02 | 0.01 | 0.02 | 0.02 | 0.02 |
| individual | | | | | | |
| unspecified | | | | | | |
| impurity | | | | | | |
| Total | 0.04 | 0.04 | 0.06 | 0.05 | 0.07 | 0.05 |
| impurities | | | | | | |

TABLE 8
Stability study results of API with Labrasol at various storage conditions

| Batch No. | APAL1007E | | | | | |
|---|---|---|---|---|---|---|
| | | 15 Days | 30 Days | 30 Days | 30 Days | 30 Days |
| Stability | | 60° C. | 25° C. | 25° C. | 40° C. | 40° C. |
| time | | Close | Close | Open | Open | Close |
| point | Initial | Vial | Vial | Vial | Vial | Vial |
| Des- | Color- | Color- | Color- | Color- | Color- | Color- |
| cription | less | less | less | less | less | less |
| | solution | solution | solution | solution | solution | solution |

Relates Substances (% w/w)

| | | | | | | |
|---|---|---|---|---|---|---|
| Acid | 0.02 | 0.03 | 0.03 | 0.02 | 0.03 | 0.03 |
| Impurity | | | | | | |
| Any | 0.03 | 0.42 | 0.14 | 0.19 | 0.51 | 0.30 |
| individual | | | | | | |
| unspecified | | | | | | |
| impurity | | | | | | |
| Total | 0.13 | 1.35 | 0.36 | 0.41 | 1.65 | 0.63 |
| impurities | | | | | | |

TABLE 9
Stability study results of API with Neotame at various storage conditions

| Batch No. | APAL1007F | | | | | |
|---|---|---|---|---|---|---|
| | | 15 Days | 30 Days | 30 Days | 30 Days | 30 Days |
| | | 60° C. | 25° C. | 25° C. | 40° C. | 40° C. |
| Stability time | | Close | Close | Open | Open | Close |
| point | Initial | Vial | Vial | Vial | Vial | Vial |
| Description | White | White | White | White | White | White |
| | color | color | color | color | color | color |
| | powder | powder | powder | powder | powder | powder |

Related Substances (% w/w)

| | | | | | | |
|---|---|---|---|---|---|---|
| Acid Impurity | 0.02 | 0.02 | 0.02 | 0.02 | 0.02 | 0.02 |
| Any individual | 0.02 | 0.03 | 0.01 | 0.01 | 0.02 | 0.02 |
| unspecified | | | | | | |
| impurity | | | | | | |
| Total | 0.08 | 0.07 | 0.06 | 0.06 | 0.07 | 0.06 |
| impurities | | | | | | |

TABLE 10
Stability study results of API with Silicon dioxide at various storage
conditions

| Batch No. | APAL1007G | | | | | |
|---|---|---|---|---|---|---|
| | | 15 Days | 30 Days | 30 Days | 30 Days | 30 Days |
| | | 60° C. | 25° C. | 25° C. | 40° C. | 40° C. |
| Stability time | | Close | Close | Open | Open | Close |
| point | Initial | Vial | Vial | Vial | Vial | Vial |
| Description | White | White | White | White | White | White |
| | color | color | color | color | color | color |
| | fluffy | fluffy | fluffy | fluffy | fluffy | fluffy |
| | powder | powder | powder | powder | powder | powder |

Related Substances (% w/w)

| | | | | | | |
|---|---|---|---|---|---|---|
| Acid Impurity | 0.02 | 0.02 | 0.02 | 0.02 | 0.02 | 0.02 |
| Any individual | — | 0.01 | 0.02 | 0.02 | 0.02 | 0.03 |
| unspecified | | | | | | |
| impurity | | | | | | |
| Total | 0.02 | 0.03 | 0.07 | 0.06 | 0.07 | 0.07 |
| impurities | | | | | | |

TABLE 11
Stability study results of API with Orange Flavor at various storage
conditions

| Batch No. | APAL1007H | | | | | |
|---|---|---|---|---|---|---|
| | | 15 Days | 30 Days | 30 Days | 30 Days | 30 Days |
| | | 60° C. | 25° C. | 25° C. | 40° C. | 40° C. |
| Stability time | | Close | Close | Open | Open | Close |
| point | Initial | Vial | Vial | Vial | Vial | Vial |
| Description | White | # | # | # | # | # |
| | turbid | | | | | |
| | solution | | | | | |

Related Substances (% w/w)

| | | | | | | |
|---|---|---|---|---|---|---|
| Acid Impurity | — | 0.03 | 0.02 | 0.02 | 0.02 | 0.02 |
| Any individual | — | 0.02 | 0.01 | 0.01 | 0.02 | 0.02 |
| unspecified | | | | | | |
| impurity | | | | | | |
| Total impurities | — | 0.10 | 0.04 | 0.04 | 0.07 | 0.05 |

#due to heat exposure, the solution was evaporated and therefore there was no solution/particle shown in the vial

TABLE 12
Stability study results of the composite sample (API, Phosal 50 PG,
MCT, Ethanol, Labrasol, Neotame, Orange flavor and Silicon
dioxide) at various storage conditions

| Batch No. | APAL1007I | | | | | |
|---|---|---|---|---|---|---|
| | | 15 Days | 30 Days | 30 Days | 30 Days | 30 Days |
| | | 60° C. | 25° C. | 25° C. | 40° C. | 40° C. |
| Stability time | | Close | Close | Open | Open | Close |
| point | Initial | Vial | Vial | Vial | Vial | Vial |
| Description | Yellow | Yellow | Yellow | Yellow | Yellow | Yellow |
| | color | color | color | color | color | color |
| | solution | solution | solution | solution | solution | solution |

Related Substances (% w/w)

| | | | | | | |
|---|---|---|---|---|---|---|
| Acid Impurity | 0.03 | 0.04 | 0.03 | 0.03 | 0.02 | 0.03 |
| Any individual | 0.02 | 0.04 | 0.02 | 0.02 | 0.12 | 0.03 |
| unspecified | | | | | | |
| impurity | | | | | | |
| Total impurities | 0.06 | 0.16 | 0.08 | 0.09 | 0.21 | 0.12 |

From the above stability study results of the drug-excipient compatibility studies it can be concluded that there was no significant change observed in the physico-chemical parameters after 15 days at 60° C. and after 30 days at 40° C./75% RH and at 25° C./60% RH.

### Example-4: Preparation of the Aqueous Solution of Apixaban (without Solubility Enhancer)

| S. | | Quantity | | Functional |
|---|---|---|---|---|
| No. | Ingredients | mg/mL | % w/v | Category |
| 1 | Apixaban | 1.00 | 0.1 | Active |
| | | | | Pharmaceutical |
| | | | | Ingredient |
| 2 | Methyl paraben | 0.77 | 0.077 | Preservative |
| 3 | Propyl paraben | 0.085 | 0.0085 | Preservative |
| 4 | Citric acid anhydrous | 0.127 | 0.0127 | Buffer |
| 5 | Trisodium citrate | 0.057 | 0.0057 | Buffer |
| | dihydrate | | | |
| 6 | Sucralose | 4.00 | 0.4 | Sweetener |
| 7 | Orange flavor | 12.5 | 1.25 | Flavour |
| 8 | Propylene glycol | 100-800 | 10-80 | Solvent/ |
| | | | | co-solvent |
| 9 | Purified water | Q.S. to 1 mL | Q.S. to 100% | Vehicle |

### Example-5: Preparation of the Aqueous Solution of Apixaban with Solubility Enhancer-HPBCD

| S. | | Quantity | | Functional |
|---|---|---|---|---|
| No. | Ingredients | mg/mL | % w/v | Category |
| 1 | Apixaban | 0.5-1.00 | 0.05-0.1 | Active |
| | | | | Pharmaceutical |
| | | | | Ingredient |
| 2 | Methyl paraben | 0.2-2.0 | 0.02-0.2 | Preservative |
| 3 | Propyl paraben | 0.02-0.2 | 0.002-0.02 | Preservative |
| 4 | Neotame | 0.1-0.5 | 0.01-0.05 | Sweetener |
| 5 | Hydroxypropyl | 10.0-40.0 | 1.0-4.0 | Solubility |
| | beta-cyclodextrin | | | enhancer |
| | (Kleptose) | | | |
| 6 | Glycerin | 60.0-240.0 | 6.0-24.0 | Solvent |
| 7 | Propylene Glycol | 30.0-120.0 | 3.0-12.0 | Solvent |
| 8 | Polyethylene | 10.0-40.0 | 1.0-4.0 | Solvent |
| | Glycol 400 | | | |
| 9 | Oxalic acid | In a quantity | | pH adjusting |
| | | sufficient to | | agent |
| | | adjust the | | |
| | | pH of the | | |
| | | solution | | |
| | | about 4.0 | | |
| 10 | Orange flavor | 0.2-2.0 | 0.02-0.2 | Flavour |
| 11 | Purified water | Q.S. to 1 mL | Q.S. to 100% w/v | Vehicle |

### Example-6: Preparation of the Aqueous Solution of Apixaban with Solubility Enhancer-HPBCD

| S. | | Quantity | | Functional |
|---|---|---|---|---|
| No. | Ingredients | mg/ml | % w/v | Category |
| 1 | Apixaban | 0.5-1.00 | 0.05-0.1 | Active |
| | | | | Pharmaceutical |
| | | | | Ingredient |
| 2 | Methyl paraben | 0.2-2.0 | 0.02-0.2 | Preservative |
| 3 | Propyl paraben | 0.02-0.2 | 0.002-0.02 | Preservative |
| 4 | Neotame | 0.1-0.5 | 0.01-0.05 | Sweetener |
| 5 | Hydroxypropyl beta- | 10.0-40.0 | 1.0-4.0 | Solubility |
| | cyclodextrin | | | enhancer |
| | (Kleptose) | | | |
| 6 | Ethanol* | 50.0 | 5.0 | Solvent |
| 7 | Glycerin | 60.0-240.0 | 6.0-24.0 | Solvent |
| 8 | Propylene Glycol | 30.0-120.0 | 3.0-12.0 | Solvent |
| 9 | Polyethylene | 10.0-40.0 | 1.0-4.0 | Solvent |
| | Glycol 400 | | | |
| 10 | Oxalic acid | In a quantity sufficient | | pH adjusting |
| | | to adjust the | | agent |
| | | pH of the solution | | |
| | | about 4.0 | | |
| 11 | Orange flavor | 0.2-2.0 | 0.02-0.2 | Flavour |
| 12 | Purified water | Q.S. to 1 mL | Q.S. to 100% | Vehicle |

*Final formulation does not contain ethanol.

### Example-7: Preparation of the Aqueous Solution of Apixaban with Solubility Enhancer-Povidone (Polyvinylpyrrolidone)

| S. | | Quantity | | Functional |
|---|---|---|---|---|
| No. | Ingredients | mg/mL | % w/v | Category |
| 1 | Apixaban | 0.50-1.00 | 0.05-0.1 | API |
| 2 | Propylene glycol | 70.0-280.0 | 7.0-28.0 | Solvent |
| 3 | Polyethylene glycol 400 | 25.0-100.0 | 2.5-10.0 | Solvent |
| 4 | Povidone (PVP K30) | 20.0-80.0 | 2.0-8.0 | Solubility |
| | | | | enhancer |
| 5 | Sodium citrate | 0.03-0.10 | 0.003-0.01 | Buffer |
| 6 | Citric acid anhydrous | 0.05-0.3 | 0.005-0.03 | Buffer |
| 7 | Benzoic acid | 1.0-4.0 | 0.1-0.4 | Preservative |
| 8 | Glycerin | 100.0-400.0 | 10.0-40.0 | Solvent |
| 9 | Neotame | 0.1-0.5 | 0.01-0.05 | Sweetener |
| 10 | Orange flavor | 0.2-2.0 | 0.02-0.2 | Flavor |
| 11 | Purified water | Q.S. to 1 mL | Q.S. to 100% | Vehicle |

### Table-13: Stability Study Results of the Aqueous Solutions of Examples 5-7

The apixaban aqueous oral solutions prepared according to Examples 5-7 were tested further at T₀ time point. The results are summarized in the table below.

| Batch No. | APAL1016 | APAL1021 | APAL1025 | APAL1029 | APAL1030 |
|---|---|---|---|---|---|
| Apixaban (mg/mL) | 0.5 | 0.5 | 0.5 | 0.5 | 0.5 |
| Solubility enhancer | Povidone | HPBCD | HPBCD | Povidone | HPBCD |
| Description | Light yellow | Colorless | Colorless | Colorless | Colorless |
| | colored | solution | solution | solution | solution |
| | solution | | | | |
| % w/w Assay of Apixaban | 102.70 | 101.30 | 98.00 | 99.10 | 98.90 |

| | |
|---|---|
| pH | Between about 3.0 and about 6.0 |

% w/w RELATED SUBSTANCES

| | | | | | |
|---|---|---|---|---|---|
| Impurity-A | ND | ND | ND | ND | ND |
| Impurity-B (Acid Impurity) | 0.03 | 0.02 | 0.03 | 0.02 | 0.03 |
| Impurity-C | 0.01 | 0.03 | ND | 0.01 | ND |
| Impurity-D | ND | ND | ND | ND | ND |
| Impurity-E | ND | ND | ND | ND | ND |
| Impurity-G | ND | ND | ND | ND | ND |
| Impurity-I | ND | ND | ND | ND | ND |
| Impurity-J | ND | ND | ND | ND | ND |
| Unknown impurity at RRT-0.55 | 0.02 | ND | ND | 0.01 | ND |
| Unknown impurity at RRT-0.6 | ND | ND | ND | ND | ND |
| Unknown impurity at RRT-1.10 | 0.01 | ND | 0.02 | ND | ND |
| Unknown impurity at RRT-1.37 | ND | ND | ND | ND | ND |
| Total impurities | 0.07 | 0.05 | 0.05 | 0.04 | 0.03 |

### Example-8: Preparation of the Non-Aqueous Solution of Apixaban with Viscosity Modifying Agent

Batch No: APAL1033

| | Quantity | |
|---|---|---|
| Ingredients | mg/mL | % w/v |
| Apixaban | 1.00 | 0.1 |
| Labrasol | 180 | 18 |
| Phosal 50 PG | 600 | 60 |
| Neotame | 1.00 | 0.1 |
| Orange Flavour | 2.00 | 0.2 |
| Methacrylic acid-Methyl methacrylate | 2.00 | 0.2 |
| copolymer (1: 2) (Eudragit-S-100) | | |
| Medium Chain Triglycerides | Q.S. to 1.0 ml | Q.S. to 100% |

TABLE 14
Dissolution data of Batch No. APAL1033 in various
dissolution media

| | | | | F2 |
|---|---|---|---|---|
| | Time | % Drug release | | value of |
| Dissolution | points | Reference | Test | the test |
| conditions | (min) | product | product | product |
| Media: pH | 0 | 0 | 0 | 67 |
| 4.5 Acetate | 5 | 32 | 39 | |
| Buffer + 0.05% | 10 | 73 | 64 | |
| SLS | 15 | 83 | 80 | |
| Volume: 900 | 20 | 87 | 87 | |
| mL, RPM 75, | 30 | 90 | 91 | |
| Apparatus: | 45 | 92 | 92 | |
| USP Type-II | 60 | 93 | 93 | |
| | 0 | 0 | 0 | 50 |
| Media: 0.1N | 5 | 17 | 43 | |
| HCl + 0.05% | 10 | 58 | 57 | |
| SLS | 15 | 72 | 68 | |
| Volume: 900 | 20 | 77 | 74 | |
| mL, RPM 75, | 30 | 81 | 79 | |
| Apparatus: | 45 | 83 | 82 | |
| USP Type-II | 60 | 83 | 84 | |
| OGD Media: | 0 | 0 | 0 | 60 |
| pH 6.8 | 5 | 34 | 41 | |
| Phosphate | 10 | 77 | 65 | |
| Buffer + 0.05% | 15 | 86 | 80 | |
| SLS | 20 | 90 | 88 | |
| Volume: 900 | 30 | 95 | 92 | |
| mL, RPM 75, | 45 | 95 | 92 | |
| Apparatus: | 60 | 96 | 92 | |
| USP Type-II | | | | |

TABLE 15
Physico-chemical parameters of Batch No. APAL1033

| Parameter | Results |
|---|---|
| Description | Light yellow solution |
| % Assay of Apixaban | 99.70 |
| Ethanol content | 1.1% |

Related Substances (% w/w)

| | |
|---|---|
| Acid Impurity | 0.04 |
| Single maximum unspecified | 0.05 |
| impurity | |
| Total impurities | 0.09 |

### Example-9: Preparation of the Non-Aqueous Solution of Apixaban with Viscosity Modifying Agent

Batch No: APAL1034

| | Quantity | |
|---|---|---|
| Ingredients | mg/mL | % w/v |
| Apixaban | 1.00 | 0.1 |
| Labrasol | 180 | 18 |
| Phosal 50 PG | 600 | 60 |
| Neotame | 1.00 | 0.1 |
| Orange Flavour | 2.00 | 0.2 |
| Methacrylic acid-Methyl methacrylate | 2.00 | 0.2 |
| copolymer (1: 1) (Eudragit-L-100) | | |
| Medium Chain Triglycerides | Q.S. to 1.0 ml | Q.S. to 100% |

TABLE 14
Dissolution data of Batch No. APAL1034 in various
dissolution media

| | Time | % Drug release | | F2 value of |
|---|---|---|---|---|
| Dissolution | points | Reference | Test | the test |
| conditions | (min) | product | product | product |
| Media: pH | 0 | 0 | 0 | 59 |
| 4.5 Acetate | 5 | 32 | 41 | |
| Buffer + 0.05% | 10 | 73 | 61 | |
| SLS | 15 | 83 | 75 | |
| Volume: 900 | 20 | 87 | 83 | |
| mL, RPM 75, | 30 | 90 | 89 | |
| Apparatus: | 45 | 92 | 90 | |
| USP Type-II | 60 | 93 | 92 | |
| Media: 0.1N | 0 | 0 | 0 | 48 |
| HCl + 0.05% | 5 | 17 | 43 | |
| SLS | 10 | 58 | 61 | |
| Volume: 900 | 15 | 72 | 68 | |
| mL, RPM 75, | 20 | 77 | 72 | |
| Apparatus: | 30 | 81 | 75 | |
| USP Type-II | 45 | 83 | 75 | |
| | 60 | 83 | 77 | |
| OGD Media: | 0 | 0 | 0 | 49 |
| pH 6.8 | | | | |
| Phosphate | 5 | 34 | 38 | |
| Buffer + 0.05% | 10 | 77 | 58 | |
| SLS | 15 | 86 | 72 | |
| Volume: 900 | 20 | 90 | 81 | |
| mL, RPM 75, | 30 | 95 | 88 | |
| Apparatus: | 45 | 95 | 89 | |
| USP Type-II | 60 | 96 | 90 | |

TABLE 15
Physico-chemical parameters of Batch No. APAL1034

| Parameter | Results |
|---|---|
| Description | Light yellow solution |
| %Assay of Apixaban | 98.70 |
| Ethanol content | 1.1% |

Related Substances (% w/w)

| | |
|---|---|
| Acid Impurity | Not detected |
| Single maximum unspecified | 0.05 |
| impurity | |
| Total impurities | 0.07 |

### Example-10: Bioequivalence Study for the Non-Aqueous Apixaban Oral Solution as Prepared in Example-2 in Accordance with the Present Invention

The bio-equivalence study of the non-aqueous oral solution of apixaban as described in the above mentioned Example-2 (test formulation, ‘T’) was carried out with the Apixaban reference drug product-Eliquis® tablets (manufactured by Bristol Myers Squibb Company, reference formulation, ‘R’) in the human subjects under fed conditions. The results of said bio-equivalence study has been summarized in the table below.

TABLE 16
Summary of results for Apixaban BE study (Test v Reference)

| | | | |
|---|---|---|---|
| | | AUC(0-t) | AUC(0-inf) |
| Parameter | Cmax | (h*ng/ml) | (h*ng/ml) |

Geometric LSM

| | | | |
|---|---|---|---|
| Test-T | 130.88 | 1243.59 | 1259.42 |
| Reference-R | 132.77 | 1171.52 | 1184.59 |
| Geometric LSM Ratio (%) | 98.57 | 106.15 | 106.32 |
| 90% Confidence | 93.70-103.70 | 101.64-110.86 | 101.84-110.99 |
| Interval (%) | | | |

Two products are bioequivalent if the 90% Confidence Interval of the relative mean Cₘₐₓ, AUC₍₀₋ₜ₎ and AUC_{(0-∞)} of the test formulation to reference formulation (e.g. the brand product) is within 80% to 125%. From above data it can be seen that the 90% confidence interval of the relative mean Cₘₐₓ, AUC₍₀₋ₜ₎ and AUC_{(0-∞)} of the test formulation to reference formulation is within 80% to 125% and therefore the non-aqueous oral solution of apixaban of the present invention is found to be bio-equivalent to the Eliquis tablets.

### Examples 11-12: Preparation of Aqueous Oral Solution of Apixaban with Solubility Enhancing Agent

| | Example-11 | | Example-12 | |
|---|---|---|---|---|
| | Quantity | Quantity | Quantity | Quantity |
| Ingredient | ( mg/mL) | (% w/v) | ( mg/mL) | (% w/v) |
| Apixaban | 0.5 | 0.05 | 0.5 | 0.05 |
| Hydroxypropyl beta- | 20 | 2.0 | — | — |
| cyclodextrin (Kleptose) | | | | |
| Polyvinylpyrrolidone | — | — | 40 | 4.0 |
| (PVP/Povidone) | | | | |
| Oxalic acid | In a quantity sufficient | | — | — |
| | to adjust the pH | | | |
| | of the solution | | | |
| | about 3.0 to about 7.0 | | | |
| Citric acid anhydrous | — | — | 0.127 | 0.0127 |
| Sodium citrate | — | — | 0.057 | 0.0057 |
| Benzoic acid | — | — | 2.13 | 0.213 |
| Ethanol | 50 | 5.0 | — | — |
| Glycerin | 300 | 30.0 | 300 | 30.0 |
| Propylene glycol | 200 | 20.0 | 200 | 20.0 |
| Polyethylene glycol-400 | 80 | 8.0 | 80 | 8.0 |
| Water | Q.S. to | Q.S. to | Q.S. to | Q.S. to |
| | 1 mL | 100% | 1 mL | 100% |

TABLE 17
Stability study results of the aqueous solutions of Examples 11

| | | 25° C./60% RH | | 30° C./65% RH | | 40° C./75% RH | |
|---|---|---|---|---|---|---|---|
| Parameter | Initial | 3 Months | 6 Months | 3 Months | 6 Months | 3 Months | 6 Months |
| Description | Colorless | Colorless | Colorless | Colorless | Colorless | Colorless | Colorless |
| | solution | solution | solution | solution | solution | solution | solution |
| % w/w Assay of | 98.0 | 99.5 | 99.6 | 99.5 | 98.7 | 98.4 | 98.9 |
| Apixaban | | | | | | | |
| % Ethanol | 48.3 | 49.2 | 48.5 | 49.4 | 48.1 | 49.3 | 49.0 |
| content | | | | | | | |

Related Substances (% w/w)

| | | | | | | | |
|---|---|---|---|---|---|---|---|
| Impurity-B | 0.03 | 0.03 | 0.04 | 0.03 | 0.04 | 0.04 | 0.06 |
| (Acid) | | | | | | | |
| Impurity-C | Not | 0.04 | 0.01 | 0.03 | 0.01 | 0.02 | 0.05 |
| (methyl ester) | detected | | | | | | |
| Any individual | 0.02 | 0.01 | 0.03 | 0.01 | 0.05 | 0.08 | 0.23 |
| unspecified | | | | | | | |
| impurity | | | | | | | |
| Total | 0.05 | 0.03 | 0.13 | 0.07 | 0.24 | 0.31 | 0.9 |
| impurities | | | | | | | |

TABLE 18
Stability study results of the aqueous solutions of Examples 12

| | | 25° C./60% RH | | 30° C./65% RH | | 40° C./75% RH | |
|---|---|---|---|---|---|---|---|
| Parameter | Initial | 3 Months | 6 Months | 3 Months | 6 Months | 3 Months | 6 Months |
| Description | Colorless | Colorless | Colorless | Colorless | Colorless | Colorless | Colorless |
| | solution | solution | solution | solution | solution | solution | solution |
| % w/w Assay of | 99.1 | 99.2 | 98.6 | 99.0 | 98.2 | 98.1 | 94.8 |
| Apixaban | | | | | | | |
| % Content of | 97.2 | 96.8 | 94.8 | 96.6 | 91.5 | 95.1 | 92.0 |
| preservative | | | | | | | |

Related Substances (% w/w)

| | | | | | | | |
|---|---|---|---|---|---|---|---|
| Impurity-B | 0.02 | 0.05 | 0.05 | 0.06 | 0.09 | 0.11 | 0.19 |
| (Acid) | | | | | | | |
| Impurity-C | 0.01 | 0.02 | 0.02 | 0.03 | 0.02 | Not | 0.01 |
| (methyl ester) | | | | | | detected | |
| Any individual | 0.01 | 0.11 | 0.14 | 0.14 | 0.26 | 0.34 | 0.43 |
| unspecified | | | | | | | |
| impurity | | | | | | | |
| Total | 0.04 | 0.25 | 0.55 | 0.52 | 0.68 | 0.88 | 0.99 |
| impurities | | | | | | | |

All the prior arts mentioned herein are incorporated by reference in their entirety.

The apixaban formulations of the present invention are suitable for use in the industry.

It should be understood that various changes and modifications to the embodiments described herein would be apparent to those skilled in the art. Such changes and modifications can be made without departing from the spirit and scope of the subject matter of the present invention and without diminishing its intended advantages. It is. therefore, intended that such changes and modifications be covered within the scope of the present invention.

1. A non-aqueous solution of apixaban suitable for oral administration comprising apixaban in an amount from about 0.5 mg/mL to about 10 mg/mL and propylene glycol in an amount less than 35% by weight based on the total volume of the formulation.

2. The non-aqueous solution of apixaban as claimed in claim 1 further comprises one or more selected from a solvent and a vehicle.

3. The non-aqueous solution of apixaban as claimed in claim 1 further comprises one or more selected from a viscosity modifying agent, a sweetener and a flavor.

4. The non-aqueous solution of apixaban as claimed in claim 2, wherein the solvent comprises Caprylocaproyl Polyoxyl-8 glycerides (Labrasol®, Labrasol® ALF) and the vehicle comprises medium chain triglycerides.

5. The non-aqueous solution of apixaban as claimed in claim 4, wherein the vehicle further comprises phosphatidylcholine, and ethanol.

6. A non-aqueous solution of apixaban suitable for oral administration comprising
about 0.5 mg/mL to about 10 mg/mL apixaban;
about 10% to less than 35% by weight propylene glycol based on the total volume of the formulation;
phosphatidylcholine;
ethanol;
caprylocaproyl Polyoxyl-8 glycerides;
medium chain tryglycerides; and
optionally a viscosity modifying agent.

7. The non-aqueous solution of apixaban as claimed in claim 6, wherein the viscosity modifying agent is selected from the group of silicone dioxide, polyacrylate and polyacrylate copolymers such as 1:1 or 1:2 copolymers of methacrylic acid-methyl methacrylate (such as Eudragit® L-100, Eudragit® S-100), celluloses and cellulose derivatives such as methyl-, ethyl- and propyl celluloses; hydroxyalkyl-celluloses, hydroxyl propyl celluloses, hydroxylpropylalkyl celluloses, carboxymethyl cellulose or its salt, polyvinyl resins, polyethylene oxide, sodium alginate or combinations thereof.

8. The non-aqueous solution of apixaban as claimed in claim 7 further comprises a sweetener and a flavor.

9. An aqueous solution of apixaban suitable for oral administration comprising
apixaban in an amount from about 0.5 mg/mL to about 2 mg/mL; and
an agent selected from a cyclodextrin derivative, polyvinylpyrrolidone, hydroxypropyl cellulose, or hydroxypropyl methylcellulose or a combination thereof,
wherein the aqueous solution is devoid of:
surfactants; and
solubilizers such as diethylene glycol monoethylether, caprylic acid capric acid polyethylene glycol glyceride, and 15-hydroxystearic acid polyethylene glycol ester.

10. The aqueous solution of apixaban as claimed in claim 9 further comprises one or more excipients selected from the group of solvents, pH adjusting agents/buffering agents, vehicles, sweetening agents, flavors, and optionally.
